(19) United States
(12) Patent Application Publication
(10) Pub. No.: US 2022/0370466 A1
(43) Pub. Date: Nov. 24, 2022

(54) REGIMENS FOR TREATING HIV INFECTIONS AND AIDS

(71) Applicant: ViiV Healthcare Company, Wilmington, DE (US)

(72) Inventors: Michael ABOUD, Brentford (GB); Pedro Cahn, Brentford (GB); Thomas Laughery, Research Triangle Park, NC (US); Dominique J. Limet, Brentford (GB); John C. Pottage, Collegeville, PA (US); Kimberly Y. Smith, Research Triangle Park, NC (US)

(21) Appl. No.: 17/664,882

(22) Filed: May 25, 2022

Related U.S. Application Data
(63) Continuation of application No. 16/631,859, filed on Jan. 17, 2020.
(60) Provisional application No. 62/535,297, filed on Jul. 21, 2017.

(51) Int. Cl. A61K 31/5365; A61P 31/18; A61K 9/20; A61K 31/513

(52) U.S. Cl. CPC A61K 31/5365; A61P 31/18; A61K 9/20; A61K 31/513

(57) ABSTRACT

Disclosed are methods for treating human immunodeficiency virus (HIV) or AIDS in a human subject using only two antiretroviral drugs, the subject being virologically suppressed and having previously followed a treatment regimen including at least three antiretroviral drugs.

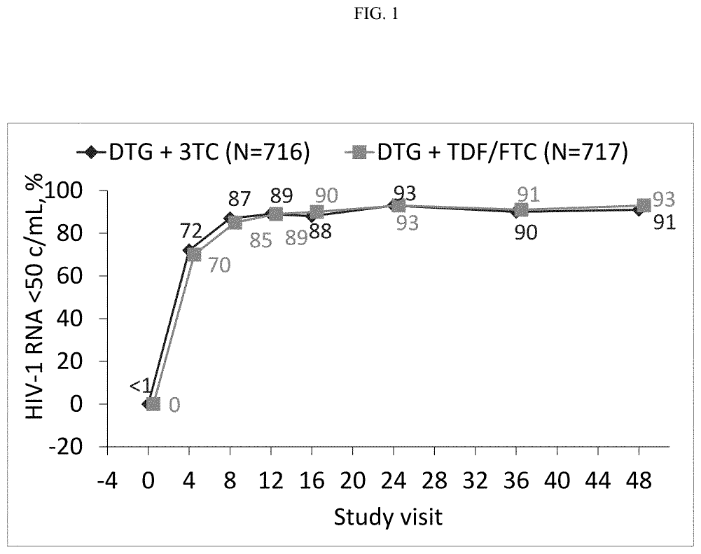

### RELATED APPLICATIONS

The present application is a Continuation of U.S. application Ser. No. 16/631,859, filed Jan. 17, 2020, which is a § 371 of International Application No. PCT/IB2018/055396, filed Jul. 19, 2018, which claims priority from U.S. Provisional Application No. 62/535,297 filed on Jul. 21, 2017, the entire contents of which is incorporated herein by reference.

### FIELD OF THE INVENTION

Methods for treating human immunodeficiency virus (HIV) or AIDS in a human subject.

### BACKGROUND OF THE INVENTION

Human immunodeficiency virus (“HIV”) infection and related diseases are a major public health problem worldwide. Human immunodeficiency virus type 1 (“HIV-1”) is a retrovirus which encodes three enzymes that are required for viral replication: reverse transcriptase, protease, and integrase. Although drugs targeting reverse transcriptase and protease are in wide use and have shown effectiveness, particularly when employed in combination, toxicity and development of resistant strains have limited their usefulness (Palella, et al., 1998 N. Engl. J. Med. 338:853-860; Richman, 2001 Nature 410:995-1001).

Antiretroviral therapy (ART) is intended to maximally suppress the HIV virus in an HIV-infected human and to stop the progression of HIV disease. Numerous clinical data sets have shown that an HIV viral load (RNA level) decreases following ART initiation and that the decrease in HIV viral load correlates with a reduced risk of AIDS progression and/or AIDS-related death. (Murray et al., The use of plasma HIV RNA as a study endpoint in efficacy trials of antiretroviral drugs1999 AIDS 13(7):797-804; Marschner et al., Use of changes in plasma levels of human immunodeficiency virus type 1 RNA to assess the clinical benefit of antiretroviral therapy, 1998 J Infect Dis. 177(1):40-47; Thiebaut et al., Clinical progression of HIV-1 infection according to the viral response during the first year of antiretroviral treatment, 2000 AIDS 14(8):971-978.) In light of these findings, HIV viral load testing is an established technique for determining treatment response and, in particular, a decreased HIV viral load is an accepted marker of a positive treatment response. (HIV Surrogate Marker Collaborative Group, Human immunodeficiency virus type 1 RNA level and CD4 count as prognostic markers and surrogate endpoints: a meta-analysis, 2000 AIDS Res Hum Retroviruses, 16(12):1123-1133.) A statistically significant change in viral load is a three-fold change, which equates to a 0.5 log₁₀ copies/mL change. Depending on the study or assay used, viral suppression in general is defined as a viral load below the level of detection, which is an HIV RNA copy number of less than 20 to 75 copies per milliliter (c/mL). Viral suppression may be defined as an HIV RNA copy number less than 50 copies per milliliter (<50 c/mL). (Damond et al., Human immunodeficiency virus type 1 (HIV-1) plasma load discrepancies between the Roche COBAS AMPLICOR HIV-1 MONITOR Version 1.5 and the Roche COBAS AmpliPrep/COBAS TaqMan HIV-1 assays, 2007 J Clin Microbiol. 45(10): 3436-3438.)

Treatment guidelines published by the United States Department of Health and Human Services provide that ART is recommended for all HIV-infected individuals (regardless of CD4 cell count) and achieving viral suppression requires the use of combination therapies (two or more drugs from at least two drug classes). (PANEL ON ANTIRETROVIRAL GUIDELINES FOR ADULTS AND ADOLESCENTS: GUIDELINES FOR THE USE OF ANTIRETROVIRAL AGENTS IN HIV-1-INFECTED ADULTS AND ADOLESCENTS. Department of Health and Human Services. Available at http://aidsinfo.nih.gov/ContentFiles/AdultandAdolescentGL.pdf Section accessed Apr. 19, 2017.) In fact, the standard course of care for a patient infected with HIV or diagnosed with AIDS is to treat them with a combination of three or more antiretroviral (ARV) agents (“combination antiretroviral therapy” (cART)). Frequently, this treatment uses two different antiretroviral agents each targeting HIV reverse transcriptase (a “backbone”) and at least one additional antiretroviral agent (an “anchor”) that is active against: an HIV protease inhibitor, an HIV non-nucleoside or non-nucleotide inhibitor of reverse transcriptase, an HIV nucleoside or nucleotide inhibitor of reverse transcriptase, an HIV integrase inhibitor, an HIV non-catalytic site (or allosteric) integrase inhibitor, or a combination thereof.

While ART has led to substantial increases in life expenctancy and quality of life for HIV-infected persons, HIV infection requires lifelong treatment. This means that as HIV-infected individuals achieve life expectancies near those or persons without HIV, HIV-infected individuals are likewise starting to receive treatment for non-HIV, common conditions such as diabetes, cardiovascular disease, arthritis, osteoporosis, or other age-associated conditions and diseases. (Zhou et al., Total Daily Pill Burden in HIV-Infected Patients in the Southern United States, 2014 AIDS PATIENT CARE and STDs 28(6): 311-317.) This increased drug burden (of HIV patients also now taking medications for HIV-unrelated indications) raises risks of drug-drug interactions and overlapping toxicities, not to mention it increases the patient's healthcare costs and dosing hassle. (Zhou et al., AIDS PATIENT CARE and STDs 28(6): 311-317.) Further, increasing medication complexity may affect treatment adherence and virologic suppression. (Zhou et al., AIDS PATIENT CARE and STDs 28(6): 311-317.)

Different combinations of antiretroviral drugs have been developed for the treatment of HIV, including dual (2 ARV drug) combination therapies. However, previously developed 2-drug therapies rapidly led to HIV treatment failure due to the emergence of resistance-associated mutations and because they did not suppress HIV well. (Vella et al., The History of Antiretroviral Therapy and of its Implementation in Resource-limited Areas of the World, 2012 AIDS 26:1231-1241; Lolekha et al., Resistance to Dual Nucleoside Reverse-Transcriptase Inhibitors in Children Infected with HIV Clade A/E, 2005 Clin. Infect. Dis 40(2): 309-312.) In fact, 2-drug HIV treatment regimens until now remain disfavored by the art at least because they are believed to pose “higher risks of resistance.” (Gilead HIV Drug Tops GSK Combo, But Rivalry ‘Intense’ With GSK Doublet, an article in INVESTOR'S BUSINESS DAILY, Feb. 14, 2017.) However, it is generally accepted in terms of long term toxicity two drug therapies is better than therapies involving three or more drugs.

For at least the reasons set forth above, there remains an unmet medical need to reduce the drug burden of patients infected with HIV or diagnosed with AIDS and to, in particular, treat them with fewer antiretroviral agents, such as with only two (2) ARVs. Further, there remains an unmet medical need for effective 2-ARV-drug HIV treatment regimens.

### SUMMARY OF THE INVENTION

The present invention provides methods and compositions for treating a subject infected with Human Immunodeficiency Virus using only two ARVs.

The present invention provides a method of treating a Human

Immunodeficiency Virus (HIV) infected adult subject who adhered to an at least 3-drug (3-drug+) AntiRetroviral Therapy (ART) regimen and is virologically suppressed, comprising administering to the subject a therapeutic dose of each of only 2 ARV drugs at a dosing interval, the 2 ARV drugs being (1) dolutegravir (DTG) or a pharmaceutically acceptable salt thereof and (2) lamivudine (3TC) or a pharmaceutically acceptable salt thereof.

The present invention provides a method of treating a Human

Immunodeficiency Virus (HIV) infected subject who adhered to a 3-drug+AntiRetroviral Therapy (ART) regimen and is virologically suppressed, comprising administering to the subject dolutegravir (DTG) or a pharmaceutically acceptable salt thereof and lamivudine (3TC) or a pharmaceutically acceptable salt thereof at a dosing interval that results in: (i) a total daily dose of between about 40 mg and about 60 mg of DTG or pharmaceutically acceptable salt thereof and (ii) a total daily dose of between about 200 mg and about 400 mg of 3TC or pharmaceutically acceptable salt thereof.

The present invention provides a method of treating a Human Immunodeficiency Virus (HIV) infection with only 2 ARV drugs, comprising administering a total daily dose of between about 40 mg and about 60 mg of dolutegravir (DTG) or a pharmaceutically acceptable salt thereof and a total daily dose of between about 200 mg and about 400 mg lamivudine (3TC) or a pharmaceutically acceptable salt thereof to an HIV infected subject selected for (1) adhering to a 2-drug regimen of DTG and 3TC, (2) being virologically suppressed, and (3) having adhered to a 3-drug+AntiRetroviral Therapy (ART) regimen and been virologically suppressed before (1).

The present invention provides a method of reducing the drug burden of a virologically suppressed, Human Immunodeficiency Virus (HIV) infected subject on a 3-drug+ AntiRetroviral Therapy (ART) regimen, comprising: administering to the subject multiple doses of a 2-drug pharmaceutical composition at a dosing interval of between about 24 and about 48 hours wherein the 2-drug pharmaceutical composition comprises a therapeutic dose of dolutegravir (DTG) or a pharmaceutically acceptable salt thereof and a therapeutic dose of lamivudine (3TC) or a pharmaceutically acceptable salt thereof.

The present invention provides a method of treating a virologically suppressed, Human Immunodeficiency Virus (HIV) infected subjects adhering to a 3-drug+ AntiRetroviral Therapy (ART) regimen, comprising the steps of: (a) administering to the subjects once daily a therapeutic dose of each of only 2 ARV drugs, the 2 ARV drugs being (1) dolutegravir (DTG) or a pharmaceutically acceptable salt thereof and (2) lamivudine (3TC) or a pharmaceutically acceptable salt thereof; (b) obtaining the copy number of HIV RNA per milliliter of a plasma sample taken from each of the subjects 21 to 27, 49 to 55, 77 to 83, 161 to 167, 245 to 251, 329 to 335, or 358 to 362 days after (a); and then (c) administering another therapeutic dose of each of the 2 ARV drugs to the subjects selected for having a no more than 50 copies of HIV RNA per milliliter of the plasma sample.

In an embodiment, a therapeutic dose of each of only 2 ARV drugs at a dosing interval is administered to an infected subject. In a further embodiment, the subject is an adult subject, for example an adult human subject. In an embodiment of the invention, the subject is virologically suppressed prior to the 2 ARV drugs are administered. In an embodiment of the invention, the subject adhered to an at least 3-drug (3-drug+) AntiRetroviral Therapy (ART) regimen prior to the 2 ARV drug composition of the present invention being administered. In an embodiment, the subject adhered to the 3-drug+ ART regimen for at least about 4 years, about 6 months, or about 3 months before the administration of the 2 ARV drugs of the present invention. In an embodiment, the subject is selected for not having chronic hepatitis B virus infection.

In an embodiment of the invention, the 3-drug+ ART regimen was a Tenofovir Alafenamide (TAF) based (TAF3D+) regimen. The present invention provides wherein the TAF3D+ regimen comprised the administration of bictegravir or a pharmaceutically acceptable salt thereof, emtricitabine or a pharmaceutically acceptable salt thereof, and TAF or a pharmaceutically acceptable salt thereof. In an embodiment of the invention, the 3-drug+ ART regimen was a bictegravir based regimen. In another embodiment, the 3-drug+ ART regimen is a combination of DTG, FTC, and TDF regimen.

In an embodiment, the 2 ARV drugs are (1) dolutegravir (DTG) or a pharmaceutically acceptable salt thereof and (2) lamivudine (3TC) or a pharmaceutically acceptable salt thereof. The present invention provides wherein, before administration, the HIV is not partially resistant or is not completely resistant to (i) DTG or pharmaceutically acceptable salt thereof, (ii) 3TC or pharmaceutically acceptable salt thereof, or (iii) both (i) and (ii). In an embodiment of the present invention, the therapeutic dose of DTG or pharmaceutically acceptable salt thereof is between about 40 mg and about 60 mg. In a further embodiment, the therapeutic dose of DTG pharmaceutically acceptable salt thereof is about 50 mg. In an embodiment of the present invention, the therapeutic dose of 3TC or pharmaceutically acceptable salt thereof is between about 200 mg and about 400 mg. In a further embodiment, the therapeutic dose of 3TC or pharmaceutically acceptable salt there of is about 300 mg. In an embodiment of the invention, the therapeutic dose of DTG or pharmaceutically acceptable salt thereof is between about 40 mg and about 60 mg and the therapeutic dose of 3TC or pharmaceutically acceptable salt thereof is between about 200 mg and about 400 mg. In an embodiment of the invention, the therapeutic dose of DTG or a pharmaceutically acceptable salt thereof is about 50 mg and the therapeutic dose of 3TC or a pharmaceutically acceptable salt thereof is about 300 mg. In an embodiment of the present invention, the dosing interval results in (i) a total daily dose of about 50 mg of DTG or pharmaceutically acceptable salt thereof and (ii) a total daily dose of about 300 mg of 3TC or pharmaceutically acceptable salt thereof. The present invention provides wherein the dose interval between therapeutic doses or total daily doses is between about 24 and about 48 hours. The present invention provides for administering the DTG or pharmaceutically acceptable salt thereof and the 3TC or pharmaceutically acceptable salt thereof once every about 24 hours. In an embodiment of the invention, the dose interval is about 24 hours.

In an embodiment of the invention, (i) DTG or pharmaceutically acceptable salt thereof, (ii) 3TC or pharmaceutically acceptable salt thereof, or (iii) both (i) and (ii) is administered orally or by injection. In an embodiment of the invention, (i) DTG or pharmaceutically acceptable salt thereof, (ii) 3TC or pharmaceutically acceptable salt thereof, or (iii) both (i) and (ii) is in a tablet or liquid formulation.

In an embodiment of the invention, the DTG or pharmaceutically acceptable salt thereof and 3TC or pharmaceutically acceptable salt thereof are co-administered. In an embodiment of the invention, one or more non-ARV agents is administered. In an embodiment, the one or more non-ARV agents are co-administered.

In an embodiment of the invention, DTG or pharmaceutically acceptable salt thereof and 3TC or pharmaceutically acceptable salt thereof are administered contemporaneous to food consumption. In some embodiments, the food comprises an at least moderate fat content.

In an embodiment of the invention, the DTG or pharmaceutically acceptable salt thereof and/or 3TC or pharmaceutically acceptable salt thereof is within a pharmaceutical composition. In an embodiment, the DTG or pharmaceutically acceptable salt thereof and 3TC or pharmaceutically acceptable salt thereof are both within a 2-drug pharmaceutical composition. A 2-drug pharmaceutical composition of the present invention may be a fixed dose composition and/or in a tablet or liquid formulation. 2-drug pharmaceutical compositions of the present invention may be administered orally or by injection. 2-drug pharmaceutical compositions of the present invention may further comprises one or more non-ARV agents.

In an embodiment of the invention, after administration, the HIV is not partially resistant or is not completely resistant to (i) DTG or pharmaceutically acceptable salt thereof, (ii) 3TC or pharmaceutically acceptable salt thereof, or (iii) both (i) and (ii).

The present invention provides wherein, after administration, the subject is virologically suppressed. In an embodiment, virologically suppressed consists of a no more than 50 copies of HIV RNA per milliliter (<50 c/mL) of the subject's plasma.

In an embodiment of the invention, the subject is infected with Human Immunodeficiency Virus Type 1 (HIV-1).

The present invention provides for administering an at least 22nd therapeutic dose of the 2-drug pharmaceutical composition to the HIV infected subject wherein the subject is virologically suppressed after the 21st dose administration; administering an at least 50th therapeutic dose of the 2-drug pharmaceutical composition to the HIV infected subject wherein the subject is virologically suppressed after the 49th dose administration; administering an at least 78th therapeutic dose of the 2-drug pharmaceutical composition to the HIV-1 infected subject wherein the subject is virologically suppressed after the 77th dose administration; administering an at least 162nd therapeutic dose of the 2-drug pharmaceutical composition to the HIV infected subject wherein the subject is virologically suppressed after the 161st dose administration; administering an at least 246th therapeutic dose of the 2-drug pharmaceutical composition to the HIV infected subject wherein the subject is virologically suppressed after the 245th dose administration; administering an at least 330th therapeutic dose of the 2-drug pharmaceutical composition to the HIV infected subject wherein the subject is virologically suppressed after the 329th dose administration; and administering an at least 358th therapeutic dose of the 2-drug pharmaceutical composition to the HIV infected subject wherein the subject is virologically suppressed after the 357th dose administration.

The present invention provides a 2-drug pharmaceutical composition for use in treating a Human Immunodeficiency Virus (HIV) infection.

The present invention provides a 2-drug pharmaceutical composition for use in treating a Human Immunodeficiency Virus (HIV) infected subject who adhered to a 3-drug+ AntiRetroviral Therapy (ART) regimen and is virologically suppressed. The present invention provides a 2-drug pharmaceutical composition for use in treating a virologically suppressed, Human Immunodeficiency Virus (HIV) infected subject adhering to a 3-drug+ AntiRetroviral Therapy (ART) regimen.

The present invention provides a 2-drug pharmaceutical composition for use in reducing the drug burden of a virologically suppressed, Human Immunodeficiency Virus (HIV) infected subject on a 3-drug+ AntiRetroviral Therapy (ART) regimen.

The present invention provides use of a 2-drug pharmaceutical composition for treating a Human Immunodeficiency Virus (HIV) infection.

The present invention provides use of a 2-drug pharmaceutical composition for treating a Human Immunodeficiency Virus (HIV) infected subject who adhered to a 3-drug+ AntiRetroviral Therapy (ART) regimen and is virologically suppressed. The present invention provides use of a 2-drug pharmaceutical composition in treating a virologically suppressed, Human Immunodeficiency Virus (HIV) infected subject adhering to a 3-drug+ AntiRetroviral Therapy (ART) regimen.

The present invention provides use of a 2-drug pharmaceutical composition for reducing the drug burden of a virologically suppressed, Human Immunodeficiency Virus (HIV) infected subject on a 3-drug+ AntiRetroviral Therapy (ART) regimen.

### DESCRIPTION OF THE FIGURE

FIGURE I. Non-Inferior Efficacy of Dolutegravir (DTG) Plus Lamivudine (3TC) vs DTG Plus Tenofovir Disoproxil Fumarate/Emtricitabine (TDF/FTC) Fixed-Dose Combination in Antiretroviral Treatment—Naive Adults With HIV-1 Infection—Week 48 Results

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, a limited number of the exemplary methods and materials are described herein.

As used in the present disclosure and claims, the singular forms “a,” “an,” and “the” include plural forms unless the context clearly dictates otherwise; i.e., “a” means “one or more” unless indicated otherwise.

The term “and/or” as used in a phrase such as “A and/or B” is intended to include “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless specified otherwise, all of the designations “A%-B%,” “A-B%,” “A% to B%,” “A to B%,” “A%-B,” “A% to B” are given their ordinary and customary meaning. In some embodiments, these designations are synonyms.

The term “comprising” encompasses “including” as well as “consisting” e.g. a composition “comprising” X may consist exclusively of X or may include something additional e.g. X+Y. The terms “comprising” and “having” when used as a transition phrase herein are open-ended whereas the term “consisting of” when used as a transition phrase herein is closed (i.e., limited to that which is listed and nothing more).

Unless specifically stated, a process comprising a step of mixing two or more components does not require any specific order of mixing. Thus components can be mixed in any order. Where there are three components then two components can be combined with each other, and then the combination may be combined with the third component, etc. Similarly, while steps of a method may be numbered (such as (1), (2), (3), etc. or (i), (ii), (iii)), the numbering of the steps does not mean that the steps must be performed in that order (i.e., step 1 then step 2 then step 3, etc.). The word “then” may be used to specify the order of a method's steps.

“For example,” “an example,” “such as,” and “e.g.” may be used interchangeably and mean to point to or relate to non-exhaustive examples.

“HIV” or “human immunodeficiency virus” each means HIV-1 or HIV-2, or any mutant, group, clinical isolate, subtype, or clade thereof.

“Adult” as used herein means at least eighteen (18) years of age. The term “adult” may therefore be used to a reference a subject who is at least 18 years of age.

“Subject(s)” or “Human” or “Human subject(s)” or “Person(s)” herein are used to refer to a Homo sapiens.

“Adhered” as used herein means to have complied with in the past or to have previously followed. For example, “adhered to a/an . . . therapy regimen” as used herein means the subject complied with or followed the therapy regimen (i.e., the subject was administered the prescribed dose at the prescribed dosing interval). As used herein, a “virologically suppressed” subject who “adhered” to an ART regimen (e.g., a “3-drug+ ART regimen” or “3-drug+ ARV regimen”) means the subject is virologically suppressed as a result of their compliance with that prior ART regimen (e.g., virologically suppressed as a result of the 3-drug+ ARV regimen).

“ART-experienced” or “antiretroviral therapy-experienced” means with regards to a human, one currently or recently being treated with one or more antiretroviral agents used to treat HIV or AIDS.

“Antiretroviral drug” or “ARV drug” as used herein means a molecule or compound classified as being a reverse transcriptase, protease, and/or integrase inhibitor. An ARV drug of the present invention may be an inhibitor of a reverse transcriptase, protease, and/or integrase of one or more species. For example, tenofovir alafenamide fumarate (TAF) is an “ARV drug” as used herein and has been assessed for the treatment of both HIV and Hepatitis B infections. Likewise, boceprevir is an “ARV drug” as used herein and has been assessed for the treatment of Hepatitis C. Similarly, atazanavir is an “ARV drug” as used herein and has been assessed for the treatment of HIV. An ARV drug as used herein includes, for example, dolutegravir, lamivudine, bictegravir, emtricitabine, tenofovir alafenamide, rilpivirine, enofovir disoproxil fumarate, atazanavir, ritonavir, darunavir, efavirenz, etravirine, lopinavir, fosamprenavir, tenofovir, tipranavir, boceprevir, elvitegravir, raltegarvir, abacavir, or the pharamaceutically acceptable salt, hydrate, or solvate thereof. An embodiment of the invention comprises a first antiviral regimen comprising and integrase inhibitor, for example, bictegarvir, dolutegravir, elvitegravir, raltegarvir, a composition comprising dolutegravir, abacavir, and 3TC (e.g. TRIUMEQ), or a composition comprising bictegravir, emtricitibine, and tenofovir alafenamide (“TAF”) (e.g., bictegravir (50 mg.), emtricitabine (200 mg.), and TAF (25 mg.)).

A “non-ARV drug agent” or “non-ARV agent” as used herein means a biologically active or inactive ingredient that is not an antiretroviral (ARV). In some embodiments of the invention, the non-ARV agent is selected from the group consisting of a pharmacokinetic enhancer, an excipient, a multivitamin, clarithromycin, erythromycin, telethromycin, antacid, calcium supplement, iron supplement, Hz-receptor antagonist, metformin, methadone, rifabutin, daclatasvir, ethinyl estradiol, midazolam, norelgestromin, carbamazepine, daclatasvir, ferrous fumarate, omeprazole, rednisone, rifampin, or combinations thereof. In some embodiments the antacid is aluminum-containing antacid, magnesium-hydroxide-containing antacid, or calcium-carbonate-containing antacid. In some embodiments the Hz-receptor antagonist is famotidine, cimetidine, nizatidine, or ranitidine.

“An at least 3-drug antiretroviral therapy” regimen or “3-drug+ ARV” regimen or “3-drug+ ART” regimen as used herein means a regimen that includes the administration of at least three (3) antiretroviral (ARV) drugs. In an embodiment of the invention, the 3-drug+ ART regimen comprises the administration of at least three of the ARV drugs selected from the group consisting of dolutegravir, lamivudine, bictegravir, emtricitabine, tenofovir alafenamide, tenofovir disoproxil fumarate, rilpivirine, enofovir disoproxil fumarate, atazanavir, ritonavir, darunavir, efavirenz, etravirine, lopinavir, fosamprenavir, tenofovir, tipranavir, boceprevir, elvitegravir, raltegarvir, abacavir, and the pharamaceutically acceptable salt, hydrate, or solvate thereof. In an embodiment of the invention, the 3-drug+ ART regimen comprises the administration of at least bictegravir, emtricitabine, and TAF. In an embodiment of the invention, the 3-drug+ ART regimen comprises the administration of at least 50 mg bictegravir, 200 mg emtricitabine, and 25 mg TAF. In another embodiment of the invention, the 3-drug+ ART regimen comprises the administration of at least 50 mg dolutegravir, 300 mg tenofovir disoproxil fumarate or 25 mg tenofovir alafenamide, and 200 mg emtricitabine.

An embodiment of the invention is provided that comprises switching to using a 2-ARV-drug an antiretroviral regimen from using an antiretroviral regimen comprising two NRTIs and one or more of an antiretroviral agent selected from the group consisting of an INI, an NNRTI, or a PI. An embodiment of a regimen of the invention is provided that comprises switching to using a 2-ARV-drug composition from using a combination comprising at least three (3) antiviral compounds selected from the group consisting of: an anti-HIV agent, an HIV protease inhibitor, an HIV non-nucleoside or non-nucleotide inhibitor of reverse transcriptase, an HIV nucleoside or nucleotide inhibitor of reverse transcriptase, an HIV integrase inhibitor, MK8591 (EFdA), bictegravir, an HIV non-catalytic site (or allosteric) integrase inhibitor, an HIV entry inhibitors (e.g., a CCR5 inhibitor, a gp41 inhibitor (i.e., a fusion inhibitor) or a CD4 attachment inhibitor (e.g., combinectin), a CXCR4 inhibitor, a gp120 inhibitor, a G6PD or an NADH-oxidase inhibitor, an HIV vaccine, a latency reversing agent (e.g., a histone deacetylase inhibitor, a proteasome inhibitor, a protein kinase C (PKC) activator, or a BRD4 inhibitor), a compound that targets HIV capsid (a “capsid inhibitor”, e.g., a capsid polymerization inhibitor or a capsid disrupting compound, an HIV nucleocapsid p7 (NCp7) inhibitor, an HIV p24 capsid protein inhibitor), a pharmacokinetic enhancer, an immune-based therapy (e.g., a Pd-1 modulator, a Pd-Ll modulator, a CTLA4 modulator, an ICOS modulator, an OX40 modulator, or the like), a toll-like receptor modulator, an IL-15 agonist, an anti-HIV antibody, a bispecific antibody or an “antibody-like” therapeutic protein (e.g., a DART, a DUOBODY, a BITE, an XmAb, a TandAb, a Fab derivative) including those targeting a HIV gp120 or gp41, combination drug for HIV, an HIV p 17 matrix protein inhibitor, an IL-13 antagonist, a peptidylprolyl cis-trans isomerase A modulator, a protein disulfide isomerase inhibitor, a complement C5a receptor antagonist, a DNA methyltransferase inhibitor, an HIV vif gene modulator, a Vif dimerization antagonist, an HIV-1 viral infectivity factor inhibitor, a TAT protein inhibitor, an HIV-1 Nef modulator, an Hck tyrosine kinase modulator, a mixed lineage kinase-3 (MLK-3) inhibitor, an HIV-1 splicing inhibitor, a Rev protein inhibitor, an integrin antagonist, a nucleoprotein inhibitor, a splicing factor modulator, a COMM domain containing protein 1 modulator, an HIV ribonuclease H inhibitor, a retrorocyclin modulator, a CDK-9 inhibitor, a dendritic ICAM-3 grabbing nonintegrin 1 inhibitor, an HIV GAG protein inhibitor, an HIV POL protein inhibitor, acomplement Factor H modulator, a ubiquitin ligase inhibitor, a deoxycytidine kinase inhibitor, a cyclin dependent kinase inhibitor, a proprotein convertase PC9 stimulator, an ATP-dependent RNA helicase DDX3X inhibitor, a reverse transcriptase priming complex inhibitor, an HIV gene therapy, a PI3K inhibitor, a compound such as those disclosed in WO 2013/006738 (Gilead Sciences), US 2013/0165489 (University of Pennsylvania), WO 2013/091096A1 (Boehringer Ingelheim), WO 2009/062285 (Boehringer Ingelheim), US20140221380 (Japan Tobacco), US 20140221378 (Japan Tobacco), WO 2010/130034 (Boehringer Ingelheim), WO 2013/159064 (Gilead Sciences), WO 2012/145728 (Gilead Sciences), WO2012/003497 (Gilead Sciences), WO2014/100323 (Gilead Sciences), WO2012/145728 (Gilead Sciences), WO 2013/159064 (Gilead Sciences), WO 2012/003498 (Gilead Sciences), and WO 2013/006792 (Pharma Resources), and other drugs for treating HIV.

“+” as used herein means “or more.” For example, “3-drug+ ARV regimen” as used herein means an antiretroviral (ARV) regimen utilizing three (3) or more (+) different ARV drugs and “3-drug+ ARV regimen” therefore encompasses a regimen utilizing four (4), five (5), six (6), seven (7), eight (8), nine (9), ten (10), etc., different ARV drugs.

A “2-drug pharmaceutical composition” herein means a pharmaceutical composition consisting of only two (2) drugs. Likewise, a “2-ARV-drug” composition herein means a composition consisting of only two (2) antiretroviral (ARV) drugs and does not include, for example a composition consisting of one (1), three (3), four (4), five (5), six (6), seven (7), eight (8), nine (9), ten (10) (or any higher integer) ARV drugs. However, a 2-ARV-drug composition may comprise one or more non-ARV drug agents. For example, “a 2-drug pharmaceutical composition” of the invention that “comprises a therapeutic dose of dolutegravir or a pharmaceutically acceptable salt thereof and a therapeutic dose of lamivudine or a pharmaceutically acceptable salt thereof” consists of (1) a therapeutic dose of dolutegravir or a pharmaceutically acceptable salt thereof, (2) a therapeutic dose of lamivudine or a pharmaceutically acceptable salt thereof, and optionally (3) one or more component that is not an ARV drug ((3) may be, for example, an excipient).

“Viral load” as used herein means the amount of virus in the referenced body fluid and is usually provided as a measurement of virus particles (such as RNA copy number) per milliliter (mL). A statistically significant change in viral load is a three-fold change, which equates to a 0.5 log₁₀ copies/mL change.

“Virologically suppressed” or “viral suppression” as used herein means the HIV ribonucleic acid (RNA) copy number per milliliter (mL) (i.e., the viral load) is less than a given threshold. Viral suppression may be defined as the viral load being below the level of detection, such as an HIV RNA copy number of less than 20 to 75 copies per milliliter (c/mL). The threshold may be defined as being, for example, between 0 and 200 copies per mL, 20 copies per mL, 50 copies per mL, 100 copies per mL, or 200 copies per mL. In a particular embodiment, the threshold is defined as being 50 copies per milliliter (mL). Therefore, viral suppression may be defined as an HIV RNA copy number per mL that is less than between 0 and 200 copies per mL, 20 copies per mL, 50 copies per mL, 100 copies per mL, or 200 copies per mL. In a particular embodiment, viral suppression is defined as an HIV RNA copy number per mL that is less than 50 copies per milliliter (<50 c/mL). The HIV RNA copy number per mL may be determined using known techniques including TaqMan 2.0 (Roche Diagnostics, Indianapolis, Ind., USA) or ABBOTT REALTIME HIV-1 VIRAL LOAD (Abbott Molecular Inc., Des Plaines, Ill., USA). In an embodiment, virologically suppressed is defined as having an HIV copy number of less than between 0 and 200 copies per mL, 20 copies per mL, 50 copies per mL, 100 copies per mL, or 200 copies per mL. Provided also as an embodiment for any copy number of the invention are each integer copy number between each end number of a copy number range. For example, a copy number range from 20 copies per mL to 50 copies per mL would also include 21, 22, 23 up to 49 copies per mL.

“Therapeutic dose” as used herein means a dose that, when administered according to a dose interval, is effective to reduce the viral load below a given threshold. In some embodiments, a therapeutic dose, of for example dolutegravir (DTG), is defined as about 40 milligrams (mg) to about 45 mg, about 40 mg to about 50 mg, about 40 mg to about 55 mg, about 40 mg to about 60 mg, about 45 mg to about 50 mg, about 45 mg to about 55 mg, about 45 mg to about 60 mg, about 50 mg to about 55 mg, about 50 mg to about 60 mg, or about 55 mg to about 60 mg. In a particular embodiment, a therapeutic dose of the invention is defined as about 50 mg. For example, about 50 mg dolutegravir (DTG). In some embodiments, a therapeutic dose, of for example dilutegravir (DTG), is defined as between 40 milligrams (mg) and 45 mg (40-45 mg), between 40 mg and 50 mg (40-50 mg), between 40 mg and 55 mg (40-55 mg), between 40 mg and 60 mg (40-60 mg), between 45 mg and 50 mg (45-50 mg), between 45 mg and 55 mg (45-55 mg), between 45 mg and 60 mg (45-60 mg), between 50 mg and 55 mg (50-55 mg), between 50 mg and 60 mg (50-60 mg), or between 55 mg and 60 mg (55-60 mg). In a particular embodiment, a therapeutic dose of the invention is defined as 50 mg. For example, 50 mg dolutegravir (DTG). Other therapeutic doses of dilutegravir can be determined or optimized using known pharmaceutical or clinical practices.

In some embodiments, a therapeutic dose, of for example lamivudine (3TC), is defined as about 200 mg to about 250 mg, about 200 mg to about 300 mg, about 200 mg to about 350 mg, about 200 mg to about 400 mg, about 250 mg to about 300 mg, about 250 mg to about 350 mg, about 250 mg to about 400 mg, about 300 mg to about 350 mg, about 300 mg to about 400 mg, or about 350 mg to about 400 mg. In a particular embodiment, a therapeutic dose of the invention is defined as about 300 mg. For example, about 300 mg lamivudine (3TC). In some embodiments, a therapeutic dose, of for example lamivudine (3TC), is defined as between 200 mg and 250 mg (200-250 mg), between 200 mg and 300 mg (200-300 mg), between 200 mg and 350 mg (200-350 mg), between 200 mg and 400 mg (200-40 mg), between 250 mg and 300 mg (250-300 mg), between 250 mg and 350 mg (250-350 mg), between 250 mg and 400 mg (250-400 mg), between 300 mg and 350 mg (300-350 mg), between 300 mg and 400 mg (300-400 mg), or between 350 mg and 400 mg (350-400 mg). In a particular embodiment, a therapeutic dose of the invention is defined as 300 mg. For example, 300 mg lamivudine (3TC). Unless stated otherwise, the term “between” herein is inclusive of the upper and lower endpoints. For example, “a therapeutic dose between 40 mg and 60 mg” includes the therapeutic doses 40 mg and 60 mg. “Exclusive” may be used to exclude the upper and lower endpoints. For example, “a therapeutic dose between 40 mg and 60 mg (exclusive)” does not include the lower end point 40 mg or the upper end point 60 mg. Other therapeutic doses of lamivudine can be determined or optimized using known pharmaceutical or clinical practices.

TABLE 1
Embodiments of dosages or dose intervals of a composition
of the invention co-administered with certain
other compositions (other non-ARVs).

| Concomitant Drug Class: | |
|---|---|
| Drug Name | Exemplary Embodiment |
| Antacids containing | Administer a composition of the invention |
| aluminum, magnesium | about 4 hours before or about 6 hours after |
| hydroxide, and/or | taking one or more antacids. |
| calcium carbonate): | |
| Cation-containing | |
| antacids^{b} or laxatives | |
| Sucralfate Buffered | |
| medications | |
| Oral calcium and iron | Administer a composition of the invention |
| supplements, such as | about 4 hours before or about 6 hours after |
| multivitamins containing | taking one or more supplements comprising |
| calcium or iron | calcium or iron. Alternatively, a |
| (non-antacid) | composition of the invention and |
| | supplements comprising calcium or iron can |
| | be taken together, for example with food. |
| H₂-Receptor Antagonists: | A composition of the invention |
| Famotidine | administered at least 12 hours after or at |
| Cimetidine | least 4 hours before an H₂-receptor |
| Nizatidine | antagonists. |
| Ranitidine | |
| Metformin^{b} | With concomitant use, limit the total daily |
| | dose of metformin to 1,000 mg either when |
| | starting metformin or a composition of the |
| | invention. When starting or stopping a |
| | composition of the invention, the metformin |
| | dose may require an adjustment, such as per |
| | a physician’s instruction. Monitoring of |
| | blood glucose when initiating concomitant |
| | use and after withdrawal of a composition |
| | of the invention. |
| Methadone | No dose adjustments are required when |
| | starting coadministered of methadone with |
| | a composition of the invention. However, |
| | clinical monitoring is recommended as |
| | methadone maintenance therapy may need |
| | to be adjusted in some patients. |

TABLE 2
Further embodiments of dosages of a 2-ARV-drug regimen of the
invention wherein dolutegravir (DTG) and certain other
compositions are administered to the subject.

| Coadministered Drug(s) | |
|---|---|
| and Dose(s) | Dose of Dolutegravir |
| Daclatasvir | Between 50 mg and 125 mg. |
| 60 mg once daily | once daily |
| Ethinyl estradiol | Between 50 mg and 125 mg. |
| 0.035 mg | twice daily |
| Metformin | Between 50 mg and 125 mg. |
| 500 mg twice daily | once daily |
| Metformin | Between 50 mg and 125 mg. |
| 500 mg twice daily | twice daily |
| Methadone | Between 50 mg and 125 mg. |
| 16 mg to 150 mg | twice daily |
| Midazolam | Between 25 mg. and 50 mg. |
| 3 mg | once daily |
| Norelgestromin | Between 50 mg and 125 mg. |
| 0.25 mg | twice daily |
| Tenofovir disoproxil fumarate | Between 50 mg and 125 mg. |
| 300 mg once daily | once daily |
| Atazanavir | Between 20 mg. and 70 mg. |
| 400 mg once daily | once daily |
| Atazanavir/ritonavir | Between 20 mg. and 70 mg. |
| 300 mg/100 mg once daily | once daily |
| Darunavir/ritonavir | Between 40 mg. and 70 mg. |
| 600 mg/100 mg twice daily | once daily |
| Efavirenz | Between 50 mg and 125 mg. |
| 600 mg once daily | once daily |
| Etravirine | Between 50 mg and 125 mg. |
| 200 mg twice daily | once daily |
| Fosamprenavir/ritonavir | Between 40 mg. and 70 mg. |
| 700 mg/100 mg twice daily | once daily |
| Lopinavir/ritonavir | Between 40 mg. and 70 mg. |
| 400 mg/100 mg twice daily | once daily |
| Tenofovir | Between 50 mg and 125 mg. |
| 300 mg once daily | once daily |
| Tipranavir/ritonavir | Between 50 mg and 125 mg. |
| 500 mg/200 mg twice daily | once daily |
| Antacid (e.g., Maalox) | Between 50 mg and 125 mg. |
| simultaneous administration | once daily |
| Antacid (e.g., Maalox) | Between 50 mg and 125 mg. |
| About 2 hours after bictegravir | once daily |
| Boceprevir | Between 50 mg and 125 mg. |
| 800 mg every 8 hours | once daily |
| Calcium carbonate 1,200 mg | Between 50 mg and 125 mg. |
| simultaneous administration (fasted) | once daily |
| Calcium carbonate 1,200 mg | Between 50 mg and 125 mg. |
| simultaneous administration (fed) | once daily |
| Calcium carbonate 1,200 mg | Between 50 mg and 125 mg. |
| About 2 h after bictegravir | once daily |
| Carbamazepine | Between 50 mg and 125 mg. |
| 300 mg twice daily | once daily |
| Daclatasvir | Between 50 mg and 125 mg. |
| 60 mg once daily | once daily |
| Ferrous fumarate 324 mg | Between 50 mg and 125 mg. |
| simultaneous administration (fasted) | once daily |
| Ferrous fumarate 324 mg | Between 50 mg and 125 mg. |
| simultaneous administration (fed) | once daily |
| Ferrous fumarate 324 mg | Between 50 mg and 125 mg. |
| About 2 h after antiretroviral | once daily |
| Multivitamin (e.g., One-A-Day) | Between 50 mg and 125 mg. |
| simultaneous administration | once daily |
| Omeprazole | Between 50 mg and 125 mg. |
| 40 mg once daily | once daily |
| Prednisone | Between 50 mg and 125 mg. |
| 60 mg once daily with taper | once daily |
| Rifampin | Between 20 mg and 125 mg. |
| 600 mg once daily | once daily |
| Rifampin | Between 20 mg and 125 mg. |
| 600 mg once daily | twice daily |
| Rifabutin | Between 50 mg and 125 mg. |
| 300 mg once daily | once daily |

The present invention provides for administering to subjects once daily a therapeutic dose of each of only 2 ARV drugs; (b) obtaining the copy number of HIV RNA per milliliter of a plasma sample taken from each of the subjects 21 to 27, 49 to 55, 77 to 83, 161 to 167, 245 to 251, 329 to 335, or 358 to 362 days after (a); and then (c) administering another therapeutic dose of each of the 2 ARV drugs to the subjects selected for having a no more than 50 copies of HIV RNA per milliliter of the plasma sample. In an embodiment of the invention, a plasma sample taken 21 days after a first administration means the plasma sample may be taken on day 22 (sample taken on day one of the 4th week of treatment), therefore a sample taken between 21 and 27 days after (a) is taken within the 4th week of treatment. In an embodiment of the invention, a plasma sample taken 49 days after a first administration means the plasma sample may be taken on day 50 (sample taken on day one of the 8th week of treatment), therefore a sample taken between 49 and 55 days after (a) is taken within the 8th week of treatment. In an embodiment of the invention, a plasma sample taken 77 days after a first administration means the plasma sample may be taken on day 78 (sample taken on day one of the 12th week of treatment), therefore a sample taken between 77 and 83 days after (a) is taken within the 12th week of treatment. In an embodiment of the invention, a plasma sample taken 161 days after a first administration means the plasma sample may be taken on day 162 (sample taken on day one of the 24th week of treatment), therefore a sample taken between 161 and 167 days after (a) is taken within the 24th week of treatment. In an embodiment of the invention, a plasma sample taken 245 days after a first administration means the plasma sample may be taken on day 246 (sample taken on day one of the 36th week of treatment), therefore a sample taken between 245 and 251 days after (a) is taken within the 36th week of treatment. In an embodiment of the invention, a plasma sample taken 329 days after a first administration means the plasma sample may be taken on day 330 (sample taken on day one of the 48th week of treatment), therefore a sample taken between 329 and 335 days after (a) is taken within the 48th week of treatment. In an embodiment of the invention, a plasma sample taken 358 days after a first administration means the plasma sample may be taken on day 359 (sample taken on day one of the 52nd week of treatment), therefore a sample taken between 358 and 362 days after (a) is taken within the 52nd week of treatment.

The present invention provides wherein the viral load is assessed on day 22 of a once daily treatment regimen (i.e., assess viral load after the 21st dose admnistration) and further provides for administering a 22nd therapeutic dose, optionally on day 22. The present invention provides wherein the viral load is assessed on day 50 of a once daily treatment regimen (i.e., assess viral load after the 49th dose admnistration) and further provides for administering a 50th therapeutic dose, optionally on day 50. The present invention provides wherein the viral load is assessed on day 78 of a once daily treatment regimen (i.e., assess viral load after the 77th dose admnistration) and further provides for administering a 78th therapeutic dose, optionally on day 78. The present invention provides wherein the viral load is assessed on day 162 of a once daily treatment regimen (i.e., assess viral load after the 161st dose admnistration) and further provides for administering a 162nd therapeutic dose, optionally on day 162. The present invention provides wherein the viral load is assessed on day 246 of a once daily treatment regimen (i.e., assess viral load after the 245th dose admnistration) and further provides for administering a 246th therapeutic dose, optionally on day 246. The present invention provides wherein the viral load is assessed on day 330 of a once daily treatment regimen (i.e., assess viral load after the 329th dose admnistration) and further provides for administering a 330th therapeutic dose, optionally on day 330. The present invention provides wherein the viral load is assessed on day 358 of a once daily treatment regimen (i.e., assess viral load after the 357th dose admnistration) and further provides for administering a 358th therapeutic dose, optionally on day 358.

An “effective amount”, such as in “a therapeutically effective amount of an ARV drug”, means an amount sufficient to cause the referenced effect or outcome. An “effective amount” can be determined empirically and in a routine manner using known techniques in relation to the stated purpose.

“Fixed dose combination” or “fixed dose composition” as used herein means a combination of at least two active drugs in a single dose form and is defined in accordance with its use in the art: see U.S. Food and Drug Administration, Guidance for Industry document entitled Fixed Dose Combination and Co-Packaged Drug Products for Treatment of HIV (May 2004); and Gautam and Saha (Fixed dose drug combinations (FDCs): rational or irrational: a view point, 2008 Br. J. Clin. Pharmacol. 65(5): 795-796).

“Dosing interval” as used herein means the time period between dose administrations (e.g., every two (2) hours) or the frequency of dose administrations over a specified period of time (e.g., twice (2 times) a day). “Dose interval” includes the first dose administration and therefore does not require an at least two dose administrations be conducted. An interval of “0”, for example a “dose interval of 0 hours,” may be used to clarify that the first dose administration is encompassed. In an embodiment of the present invention, the dosing interval is once every 6, 12, 18, 24, 30, 36, 42, or 48 hours. In an embodiment of the present invention, the dosing interval is once every 24 hours. In an embodiment of the present invention, the dosing interval is once every 48 hours. In an embodiment of the present invention, the dosing interval is once every 24 to 48 hours.

“Co-administered” as used herein means the two or more referenced molecules, compounds, drugs, agents, etc., are administered at the same time but not necessarily together in one composition. For example, in an embodiment of the invention two ARV drugs are administered at the same time (i.e., “co-administered”) but each is within a distinct (separate) pharmaceutical composition (i.e., not together). In an embodiment of the invention, “co-administration” is defined as a time interval of 0 minutes.

“Contemporaneous” is used herein when two activites may have differing durations (i.e., one takes longer than the other) and the two activities occur at overlapping periods of time. For example, a 2-ARV drug composition administered “contemporaneous to food consumption” is administered to the subject at any time at or after food consumption commences and at or before that food consumption ends (i.e., the drug is administered during a meal).

“Sequential administration” or “adjacent administration” as used herein mean that at least two of the two or more referenced molecules, compounds, drugs, agents, etc., are not co-administered and are administered in succession (one after the other). In an embodiment of the invention, at least two of the two or more drugs or agents are sequentially administered within 12, 11.5, 11, 10.5, 10, 9.5, 9, 8.5, 8, 7.5, 7, 6.5, 6, 5.5, 5, 4.5, 4, 3.5, 3, 2.5, 2, 1.5, 1, or .5 hours of each other. In a particular embodiment of the invention, the two ARV drugs are sequentially administered within 1 hour. In a particular embodiment of the invention, the two ARV drugs are sequentially administered within .5 hour (i.e., 30 minutes). As used herein “within” is inclusive such that “within 1 hour,” for example, includes 1 hour and times less than 1 hour.

“Dose regimen” or “regimen” as used herein means the dose and dose interval (i.e., refers to administering an amount of a drug and frequency of doses, respectively). In some embodiments, “dose regimen” may also refer to formulation, route of administration, and/or treatment duration.

“Multiple doses” as used herein means at least two (2) doses and therefore encompasses, for example, 3, 4, 5, 6, 7, 8, 9, 10 (and any higher integer) doses.

“Total daily dose” as used herein means the total amount (e.g., in milligrams (mg)) of a referenced molecule, compound, drug, combination, or composition that is administered to a subject in one (1) day (i.e., in one 24 hour period of time). A “total daily dose” encompasses any number of administrations. So, for example, if a total daily dose of 50 mg dolutegravir (DTG) is administered at a dose interval of every 24 hours, both one administration of 50 mg DTG every 24 hours and two administrations of 25 mg DTG each (totaling 50 mg) within 24 hours are encompassed, unless specified otherwise.

“Pharmaceutically acceptable” as used herein means capable of administration to a subject without undesirable physiological effects. For example, “pharmaceutically acceptable salt thereof” encompasses solvates, polymorphs, and/or salts of the referenced compound.

“About” or “approximately” mean within the margins of error of the field, art, or subject matter it refers to. When the terms “about” or “approximately” are used in conjunction with a numerical range, it modifies that range by extending the boundaries above and below the numerical values set forth. For example, “between about 0.005 to 0.006 means the boundaries of the numerical range extend below 0.005 and above 0.006 so that the particular value in question achieves the same functional result as that within the range 0.005 to 0.006 μM. For example, “about” and “approximately” can mean within 1 or more than 1 standard deviation as per the practice in the art. Alternatively, “about” and “approximately” can mean a range of up to 20%, preferably up to 10%, more preferably up to 5%, and more preferably up to 1% of a given value.

“Year” as used herein means the period of 365 days. “Leap year” as used herein means the period of 366 days. “Month” as used herein means the period of thirty (30) days. “Calendar month” as used herein means one of January, February, March, April, May, June, July, August, September, October, November, and December (i.e., “calendar month” is not limited by a period of 28, 29, 30, or 31 days). A “calendar year” as used herein means all of January, February, March, April, May, June, July, August, September, October, November, and December (i.e., 12 calendar months). “Week” as used herein means the period of seven (7) days. “Day” as used herein means the period of 24 hours. “Hour” as used herein means the period of sixty (60) minutes.

“Body fluid” or “biolocial specimen (biospecimen)” or “sample” are used herein to broadly reference biological materials from a subject and include, for example, blood (e.g., blood sample), urine (e.g., urine sample), blood plasma, cerebrospinal fluid, semen, synovial fluid, saliva, mucus, vaginal lubrication, amniotic fluid, tears, interstitial fluid, serum, breast milk, lumph, human feces, gastric acid, bile, pus, phlegm, and earwax. “Plasma” as used herein means blood plasma.

“Moderate fat content,” as in food comprising an at least “moderate fat content,” herein means that, of the food's total calories, about 30% are calories from fat. As used herein a “moderate fat meal” is a meal of about 600 kilocalories (kcal) with about 30% calories from fat. “High fat content,” as in food comprising an at least “high fat content,” herein means that, of the food's total calories, about 53% are calories from fat. As used herein a “high fat meal” is a meal of about 870 kilocalories (kcal) with about 53% calories from fat. “Low fat content,” as in food comprising an at least “low fat content,” herein means that, of the food's total calories, about 7% are calories from fat. As used herein a “low fat meal” is a meal of about 300 kilocalories (kcal) with about 7% calories from fat. (Song et al., Effect of Food on the Pharmacokinetics of the Integrase Inhibitor Dolutegravir, 2012 Antimicrob. Agents Chemother. 56(3): 1627-1629)

Also provided is an embodiment that is a regimen of the invention or composition of the invention administered to or used to treat an ART-experienced subject. A certain embodiment provides that this subject is also virologically suppressed.

Regimens of the invention and compositions of the invention are used to treat subjects infected with wild type or mutant HIV or virus comprising an HIV integrase homolog.

An embodiment of the invention provides a composition of the invention administered to a subject infected wild-type HIV-1 or HIV-2, an HIV of group M clade (subtype) A, B, C, D, E, F, G, or H or an HIV group O virus.

In some circumstances, a subject is tested to assess a degree of renal impairment or skin or tissue disorders. In some circumstances, a subject is selected for not having chronic hepatitis B virus infection before an administration of a 2-ARV-drug pharmaceutical composition to that subject.

An embodiment of the invention provides that in a subject (1) co-infected with hepatitis C or hepatitis B and HIV and (2) is determined to have a higher incidence of liver chemistry elevations (grade 1) observed compared to those who were not co-infected with either hepatitis virus, a method of treatment of is provided whereby a test is performed to determine HIV infection and hepatitis C infection and/or hepatitis B infection, either prior to or during a regimen of the invention.

A further embodiment provides discontinuing the use of a composition of the invention where a subject develops a rash, atopic dermatitis, or diarrhea following administration of such composition.

### Resistant Strains

A “partially resistant” strain herein is an HIV strain comprising one or more polynucleotide variations (e.g., mutations) which result in the HIV strain's reverse transcriptase, protease, and/or integrase being only partially inhibited by the referenced ARV drug (i.e., the referenced ARV drug only partially inhibits the HIV strain's reverse transcriptase, protease, and/or integrase). A “completely resistant” strain herein is an HIV strain comprising one or more polynucleotide variations (e.g., mutations) which result in the HIV strain's reverse transcriptase, protease, and/or integrase not being inhibited by the referenced ARV drug (i.e., the referenced ARV drug does not inhibit the HIV strain's reverse transcriptase, protease, and/or integrase). Resistance may be determined using known techniques including, for example, Sanger sequencing (Sanger et al., DNA Sequencing with Chain-terminating Inhibitors, 1977 PNAS 74(12): 5463-5467).

In an embodiment the human immunodeficiency virus is not resistant to either antiretroviral component of the 2-ARV-drug composition of the invention or is partially resistant to either or both of the antiretroviral component(s), or comprises an integrase resistant to an integrase inhibitor, such as elvitegravir or raltegravir.

An embodiment of the invention comprises a 2-ARV-drug composition used to treat a patient infected with a mutant HIV, such an HIV-1 mutant or and HIV-2 mutant, an HIV-1 or HIV-2 mutant comprising an NNRTI-resistant amino acid substitution, an NRTI-resistant amino acid substitution, a PI-resistant amino acid substitution, or an integrase inhibitor-resistant amino acid substiution, a raltegravir-resistant amino acid substiution, an elvitegravir-resistant amino acid substiution, or an HIV comprising one or more of an integrase amino acid substitution(s) selected from the group of: G118R, S153A/F/Y, R263K, T97A, E138K, M154I, N155H, E157E/K/Q/P, L74I/M, Q95K/L/R, G140A/C/S, V151I/L/A, T66A/I/K, E92Q/V/G, Y143C/H/R, Q148H/K/R, E138A/D/K/T/G/R/Q, G163E/K/Q/R/S, G193E/R, M50I, K101E/P, V179L, Y181C/I/V, Y188L, H221Y, F227C, M230I/L, L100I/K103N, E92Q/N155H, Q148R/N155H, T66K/L74M, E92Q/N155H, G140C/Q148R, G140S/Q148H/R/K, T97A/G140S/Q148, E138/G140/Q148, A153G/N155H/S163G, E92Q/T97AN155H/S163D, E138K/Q148K, G140S/Q148R, E92Q/N155H, S147G, H51Y, L68I/V, V72A/N/T, F121C/Y, A128T, P145S, Q146I/K/L/P/R, and E170A.

An embodiment of a regimen of the invention provides administering a 2-ARV-drug composition to a human infected with certain mutant HIV viruses showing a decrease in antiviral activity of a reverse transcriptase inhihitor, such a virus comprising one or more of the following mutations: K101E, K101P, E138A, E138G, E138K, E138R, E138Q, V179L, Y181C, Y1811, Y181V, Y188L, H221Y, F227C, M230I, M230L, or L100I+K103N.

### Formulations

An embodiment of the invention provides a 2-ARV-drug pharmaceutical composition comprising one or more pharmaceutically acceptable excipients. See “non-ARV-drug agents” above. Pharmaceutically acceptable excipients for various different dosage forms are well-known in the art and include carriers, diluents, fillers, binders, lubricants, disintegrants, glidants, colorants, pigments, taste masking agents, sweeteners, flavorants, plasticizers, and any acceptable auxiliary substances such as absorption enhancers, penetration enhancers, surfactants, co-surfactants, and specialized oils. The proper excipient(s) is (are) selected based in part on the dosage form, the intended mode of administration, the intended release rate, and manufacturing reliability. Examples of common types of excipients include various polymers, waxes, calcium phosphates, sugars, among others.

Other embodiments comprise pharmaceutical compositions formulated into various types of dosage forms, for example as solutions or suspensions, or as tablets, capsules, granules, pellets or sachets for oral administration. A particularly preferred pharmaceutical composition is in the form of a solid oral dosage form, preferably tablets. The tablet is preferably a swallowable tablet. It may optionally be coated with a film coat comprising, in essence, any suitable inert coating material known in the art. The above lists of excipients and forms are not exhaustive.

A pharmaceutical composition of the present invention can be manufactured according to standard methods known in the art. Granulates according to the invention can be obtained by dry compaction or wet granulation. These granulates can subsequently be mixed with e.g. suitable disintegrating agents, glidants and lubricants and the mixture can be compressed into tablets or filled into sachets or capsules of suitable size.

Tablets can also be obtained by direct compression of a suitable powder mixture, i.e. without any preceding granulation of the excipients.

Suitable powder or granulate mixtures according to the invention are also obtainable by spray drying, lyophilisation, melt extrusion, pellet layering, coating of the active pharmaceutical ingredient or any other suitable method. The so obtained powders or granulates can be mixed with one or more suitable ingredients and the resulting mixtures can either be compressed to form tablets or filled into sachets or capsules. The above mentioned methods known in the art also include grinding and sieving techniques permitting the adjustment of desired particle size distributions.

As a further embodiment, a composition of the invention is adapted for parenteral administration, such as through intravenous administration of one or both of a composition comprising DTG and or a composition comprising 3TC. Also provided is an embodiment comprising a solid (e.g., a nanoparticulate composition) , solution, or liquid formulation for parenteral administration. Parenteral administration can be performed using a suitable device, a number of which are known.

A composition according to the present invention may be used as medicament or be used in making a medicament. It may be supplied in packs or kits.

The symbol “↓” means to lower a dosage or frequency of dosing.

The symbol “↑” means to raise a dosage or frequency of dosing.

The symbol “↔” means to keep a dosage and frequency of dosing the same.

### Dolutegravir (DTG)

“Dolutegravir” or DTG inhibits HIV integrase by binding to the integrase active site and blocking the strand transfer step of retroviral deoxyribonucleic acid (“DNA”) integration which is essential for the HIV replication cycle. DTG is an integrase strand transfer inhibitor (INSTI). Strand transfer biochemical assays using purified HIV-1 integrase and pre-processed substrate DNA resulted in IC₅₀ (Inhibitory Concentration at 50%) values of 2.7 nM (Kalama and Murphy, Dolutegravir for the Treatment of HIV, 2012 Exp. Op. Invest. Drugs 21(4): 523-530).

A chemical name of dolutegravir is (4R,12a5)-N-[(2,4-difluorophenyl)methyl]-7-hydroxy-4-methyl-6, 8-dioxo-3 ,4,12,12a-tetrahydro-2H-pyrido[5, 6]pyrazino[2, 6-b][1,3]oxazine-9-carboxamide (CAS Registry Number 1051375-16-6). Certain regimens and compositions of the inventions comprise a pharmaceutically acceptable form of dolutegravir, such as a pharmaceutically acceptable salt, hydrate and/or solvate thereof. An exemplary pharmaceutically acceptable salt of dolutegravir is dolutegravir sodium (marketed as “TIVICAY”). A sodium salt of dolutegravir and a specific crystalline form of this sodium salt or a hydrate thereof are disclosed in U.S. Pat. No. 8,624,023. Amorphous dolutegravir sodium is described in, for example, U.S. Pat. No. 9,206,197. Polymorphs, isomers, prodrugs, and esters of dolutegravir are also envisioned with respect to the present invention. In an embodiment of the invention, it may be specified that dolutegravir is used in its free form (not as salt), or specified that dolutegravir is present in a non-solvated/non-hydrated form. Unless specified otherwise, the weight (mg) of dolutegravir is based on the weight of dolutegravir in its free form.

Dolutegravir has the following structural formula of Formula I:

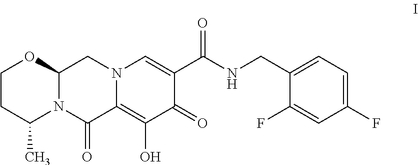 [SMILES: C[C@@H]1CCO[C@H]2Cn3cc(C(=O)NCc4ccc(F)cc4F)c(=O)c(O)c3C(=O)N12]

Methods of making dolutegravir have been described in, for example, U.S. Pat. No. 9,573,965. See also U.S. Pat. No. 8,217034 and U.S. Pregrant Publication 2016/0184332.

Dolutegravir is primarily metabolized by glucuronidation. Dolutegravir is considered to be a substrate of CYP3A4, but only to a minor extent of about 15%. Further, dolutegravir demonstrates induction or inhibition of cytochrome P450 (CYP) isozymes in vitro. See U.S. Pregrant Publication 2016/0184332.

“Dolutegravir based regimen” or “DTG based regimen” or “dolutegravir containing regimen” or “DTG containing regimen” as used herein means a regimen that includes the administration of dolutegravir or a pharmaceutically acceptable salt thereof (e.g., the administration of a pharmaceutical composition comprising dolutegravir or a pharmaceutically acceptable salt thereof).

### Lamivudine (3TC)

“Lamivudine” or 3TC is a synthetic nucleoside analogue, the active 5′-triphosphate metabolite of which (lamivudine triphosphate (3TC-TP) inhibits reverse transcriptase of at least HIV by DNA chain termination after incorporation of the nucleoside analogue. 3TC is a nucleoside reverse transcriptase inhibitor (NRTI). The antiviral activity of lamivudine against HIV-1 was assessed in a number of cell lines (including monocytes and fresh human peripheral blood lymphocytes) using standard susceptibility assays, EC₅₀ values (50% Effective Concentrations) were in the range of 0.003 to 15 μM (1 μM=0.23 mcg/mL) (see the 2013 Highlights of Prescribing Information for EPIVIR (lamivudine) Tablets and Oral Solution document at section 12.4; available at WorldWideWeb(www).viivhealthcare.com/media/32160/us_epivir.pdf).

A chemical name of lamivudine is (2R,cis)-4-amino-1-(2-hydroxymethyl-1,3-oxathiolan-5-yl)-(1H)-pyrimidin-2-one (CAS Registry Number 134678-17-4). Certain regimens and compositions of the inventions comprise a pharmaceutically acceptable form of lamivudine, such as a pharmaceutically acceptable salt , hydrate and/or solvate thereof. Polymorphs, isomers, prodrugs, and esters of lamivudine are also envisioned with respect to the present invention. Lamivudine has the following structural formula of Formula II:

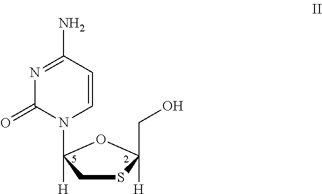

Methods of making lamivudine have been described in, for example, U.S. Pat. Nos. 5,905,082; 8,481,554; and 8,158,607.

“Lamivudine based regimen” or “3TC based regimen” or “lamivudine containing regimen” or “3TC containing regimen” as used herein means a regimen that includes the administration of lamivudine or a pharmaceutically acceptable salt thereof (e.g., the administration of a pharmaceutical composition comprising lamivudine or a pharmaceutically acceptable salt thereof).

### Tenofovir Alafenamide Fumarate (TAF)

“Tenofovir Alafenamide Fumarate” (TAF) (formerly referred to as “GS-7340”) is a fumarate salt form of “Tenofovir Alafenamide”, which is a prodrug of tenofovir (TVF). Activated, TAF is a nucleoside analog that inhibits at least HIV reverse transcriptase by DNA chain termination after incorporation of the nucleoside analogue. TAF is a nucleoside reverse transcriptase inhibitor (NRTI). TAF antiretroviral activity in CD4+ T-cells isolated from thirteen (13) persons infected with HIV demonstrated a mean EC₅₀ of 11.0 nM (median EC₅₀ of 10.2 nM), activity in monocyte-derived macrophages (MDMs) demonstrated a mean EC₅₀ of 9.7 nM (median EC₅₀ 10.3 nM) (Bam et al., Metabolism and Antiretrovial Activity of Tenofovir Alafenamide in CD4+ T-cells and Macrophages from Demographically Diverse Donors, 2014 Antiviral Ther. 19: 669-677).

A chemical name of tenofovir alafenamide fumarate (TAF) is L-alanine, N-[(S)-[[(1R)-2-(6-amino-9H-purin-9-yl)-1methylethoxy]methyl]phenoxyphosphinyl]-, 1-methylethyl ester, (2E)-2-butenedioate (2:1) (CAS Registry Number 379270-37-8). (See the 2016 Highlights of Prescribing Information for GENVOYA (elvitegravir, cobicistat, emtricitabine, and tenofovir alafenamide) Tablets, for oral use document at page 24; available at WorldWideWeb(www). accessdata.fda.gov/drugsatfda_docs/label/2016/207561s0031bl.pdf). Certain regimens and compositions of the inventions comprise a pharmaceutically acceptable form of tenofovir, such as a pharmaceutically acceptable salt, hydrate and/or solvate thereof. Certain regimens and compositions of the inventions comprise a pharmaceutically acceptable form of tenofovir alafenamide, such as a pharmaceutically acceptable salt thereof. Certain regimens and compositions of the inventions comprise a pharmaceutically acceptable form of tenofovir alafenamide fumarate, such as a pharmaceutically acceptable salt thereof. For example, tenofovir alafenamide hemifurmarate. Polymorphs, isomers, prodrugs, and esters of tenofovir (TVF) and/or of tenofovir alafenamide and/or of tenofovir alafenamide fumarate are envisioned with respect to the present invention. An alternate fumarate salt prodrug of tenofovir is, for example, tenofovir disoproxil fumarate (TDF) (see Ray et al., Tenofovir Alafenamide: A Novel Prodrug of Tenofovir for the Treatment of Human Immunodeficiency Virus, 2016 Antiviral Res. 125: 63-70). A Tenofovir alafenamide fumarate has the following structural formula of Formula III:

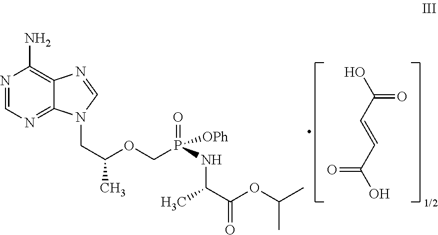

Methods of making tenofovir alafenamide and tenofovir alafenamide fumarate have been described in, for example, U.S. Pat. No. 9,487,546.

“Tenofovir based regimen” or “TFV based regimen” or “tenofovir containing regimen” or “TFV containing regimen” as used herein means a regimen that includes the administration of tenofovir or a pharmaceutically acceptable salt thereof (e.g., the administration of a pharmaceutical composition comprising tenofovir or a pharmaceutically acceptable salt thereof). For example, a regimen including the administration of emitricitabine and tenofovir disoproxil fumarate (such as that marketed as TRUVADA). For example, a regimen including the administration of tenofovir alafenamide fumarate (such as that marketed as VEMLIDY). For example, a regimen including the administration of elvitegravir, cobicistat, emtricitabine, and tenofovir alafenamide (such as that marketed as GENVOYA). For example, a regimen including the administration of emitricitabine and tenofovir alafenamide (such as that marketed as DESCOVY).

“Tenofovir alafenamide based regimen” or “tenofovir alafenamide containing regimen” as used herein means a regimen that includes the administration of tenofovir alafenamide or a pharmaceutically acceptable salt thereof (e.g., the administration of a pharmaceutical composition comprising tenofovir alafenamide or a pharmaceutically acceptable salt thereof).

A “tenofovir alafenamide fumarate based regimen” or “TAF based regimen” or “tenofovir alafenamide fumarate containing regimen” or “TAF containing regimen” as used herein means a regimen that includes the administration of tenofovir alafenamide fumarate or a pharmaceutically acceptable salt thereof (e.g., the administration of a pharmaceutical composition comprising tenofovir alafenamide fumarate or a pharmaceutically acceptable salt thereof). For examples, a regimen including the administration of tenofovir alafenamide fumarate (such as that marketed as VEMLIDY); a regimen including the administration of elvitegravir, cobicistat, emtricitabine, and tenofovir alafenamide (such as that marketed as GENVOYA); a regimen including the administration of emitricitabine and tenofovir alafenamide (such as that marketed as DESCOVY).

“TAF3D+” as used herein means an antiretroviral (ARV) therapy regimen including the administration of at least three (3) ARV drugs wherein at least one of the 3 ARV drugs is TAF.

### Bictegravir

“Bictegravir inhibits HIV integrase by binding to the integrase active site and blocking the strand transfer step of retroviral deoxyribonucleic acid (“DNA”) integration which is essential for the HIV replication cycle. Bictegravir is an integrase strand transfer inhibitor (INSTI). Strand transfer biochemical assays using purified HIV integrase and pre-processed substrate DNA resulted an IC₅₀ values of 7.5 nM (Tsiang, M. et al., Antiviral Activity of Bictegravir (GS-9883), a Novel Potent HIV-1 Integrase Strand Transfer Inhibitor with an Improved Resistance Profile, Atnimicrobial Agents and Chemotherapy, v.60: 12, 2016).

A chemical name of bictegravir is (2R,5S,13aR)-8-hidroxi-7,9-dioxo-N-[(2,4,6-trifluorofenil)metil]-2,3,4,5,7,9,13,13a-octahidro-2,5-metanopirido[1′,2′: 4,5]pirazino[2, 1-b][1,3]oxazepina-10-carboxamida (CAS Registry Number 1611493-60-7). Certain regimens and compositions of the inventions comprise a pharmaceutically acceptable form of bictegravir, such as a pharmaceutically acceptable salt , hydrate and/or solvate thereof. Bictegravir has the following structural formula of Formula IV:

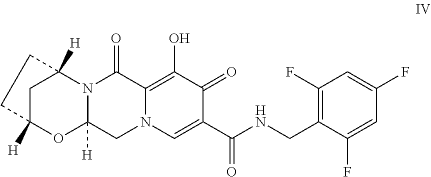 [SMILES: O=C(NCc1c(F)cc(F)cc1F)c1cn2c(c(O)c1=O)C(=O)N1[C@H]3CC[C@H](C3)O[C@@H]1C2]

A method of making bictegravir is described in the patent U.S. Pat. No. 9,216,996.

Bictegravir is metabolically cleared through certain actions of CYP3A4 and UGT1A1. In vitro studies revealed that bictegravir is a substrate of UGT1A3, UGT1A9, BCRP, and P-gp. Therefore, certain drug-drug interactions may occur. (Gallant, J. et al. Antiviral Activity, Safety, and Pharmacokinetics of Bictegravir as 10-Day Monotherapy in HIV-1—Infected Adults, 2017 Clinical Science 75(1):61-66.)

“Bictegravir based regimen” or “bictegravir containing regimen” as used herein means a regimen that includes the administration of bictegravir or a pharmaceutically acceptable salt thereof (e.g., the administration of a pharmaceutical composition comprising bictegravir or a pharmaceutically acceptable salt thereof).

### Emtricitabine (FTC)

“Emtricitabine” or FTC is a synthetic nucleoside analogue of cytidine that, in its phosphorylated active form emtricitabine 5′-triphosphate, inhibits at least HIV reverse transcriptase by being incorporated into nascent viral DNA, resulting in DNA chain termination. FTC is a nucleoside reverse transcriptase inhibitor (NRTI). The antiviral activity of emtricitabine against HIV-1 was assessed in a number of cell lines (including lymphoblastoid cell lines, MAGI-CCR5 cell line, and peripheral blood mononuclear cells), the EC₅₀ value (50% Effective Concentrations value) of emtricitabine was in the range of 0.0013-0.64 μM (0.003-0.158 μg/mL) (see the 2012 Highlights of Prescribing Information for EMTRIVA (emtricitabine) Capsule and Oral Solution document at section 12.4; available at WorldWideWeb(www). accessdatafda.gov/drugsatfda_docs/label/2012/021500s0191bl.pdf).

A chemical name of emtricitabine is 5-fluoro-1-(2R,5S)-[2-(hydroxymethyl)-1,3oxathiolan-5-yl]cytosine (CAS Registry Number 143491-57-0). Certain regimens and compositions of the inventions comprise a pharmaceutically acceptable form of emtricitabine, such as a pharmaceutically acceptable salt , hydrate and/or solvate thereof. Polymorphs, isomers, prodrugs, and esters of emtricitabine are also envisioned with respect to the present invention. Emtricitabine has the following structural formula of Formula V:

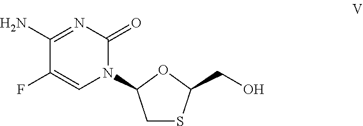 [SMILES: Nc1nc(=O)n([C@@H]2CS[C@H](CO)O2)cc1F]

Methods of making emtricitabine have been described in, for example, U.S. Pat. Nos. 7,939,660 and 7,534,885. See also U.S. Pregrant Publication 2016/0184332.

“emtricitabine based regimen” or “FTC based regimen” or “emtricitabine containing regimen” or “FTC containing regimen” as used herein means a regimen that includes the administration of emtricitabine or a pharmaceutically acceptable salt thereof (e.g., the administration of a pharmaceutical composition comprising emtricitabine or a pharmaceutically acceptable salt thereof).

Under certain circumstances, any information provided herein or derived herefrom that relates to regimen of the invention or composition of the invention may be included in a product label. Such circumstances may include, for example, requirements of a regulatory body, outcomes or data from clinical studies, or a decision of a manufacturer or other company.

All referenced patents and applications are incorporated herein by reference in their entireties.

What is claimed is:

1. A method of treating a Human Immunodeficiency Virus (HIV) infected adult subject who adhered to an at least 3-drug (3-drug+) AntiRetroviral Therapy (ART) regimen and is virologically suppressed, comprising administering to the subject a therapeutic dose of each of only 2 ARV drugs at a dosing interval, the 2 ARV drugs being (1) dolutegravir (DTG) or a pharmaceutically acceptable salt thereof and (2) lamivudine (3TC) or a pharmaceutically acceptable salt thereof.

2. The method of claim 1, wherein the 3-drug+ ART regimen was a Tenofovir Alafenamide (TAF) based (TAF3D+) regimen.

3. The method of claim 1, wherein the 3-drug+ ART regimen was a bictegravir based regimen.

4. The method of claim 2, wherein the TAF3D+ regimen comprised the administration of bictegravir or a pharmaceutically acceptable salt thereof, emtricitabine or a pharmaceutically acceptable salt thereof, and TAF or a pharmaceutically acceptable salt thereof.

5. The method of claim 1, wherein the subject adhered to the 3-drug+ ART regimen for at least about 4 years, about 6 months, or about 3 months before the administration step.

6. The method of claim 1, wherein before administration the HIV is not partially resistant or is not completely resistant to (i) DTG or pharmaceutically acceptable salt thereof, (ii) 3TC or pharmaceutically acceptable salt thereof, or (iii) both (i) and (ii).

7. The method of claim 1, wherein virologically suppressed consists of a no more than 50 copies of HIV RNA per milliliter (<50 c/mL) of the subject's plasma.

8. The method of claim 1, wherein (i) DTG or pharmaceutically acceptable salt thereof, (ii) 3TC or pharmaceutically acceptable salt thereof, or (iii) both (i) and (ii) is administered orally or by injection.

9. The method of claim 8, wherein (i), (ii), or (iii) is administered orally.

10. The method of claim 1, wherein (i) DTG or pharmaceutically acceptable salt thereof, (ii) 3TC or pharmaceutically acceptable salt thereof, or (iii) both (i) and (ii) is in a tablet or liquid formulation.

11. The method of claim 10, wherein (i), (ii), or (iii) is in a tablet formulation.

12. The method of claim 11, wherein (iii) is in a tablet formulation.

13. The method of claim 1, wherein the DTG or pharmaceutically acceptable salt thereof and 3TC or pharmaceutically acceptable salt thereof are co-administered.

14. The method of claim 1, further comprising the administration of one or more non-ARV agents.

15. The method of claim 14, wherein (1), (2), and the one or more non-ARV agents are co-administered.

16. The method of claim 1, wherein the DTG or pharmaceutically acceptable salt thereof and 3TC or pharmaceutically acceptable salt thereof are administered contemporaneous to food consumption.

17. The method of claim 16, wherein the food comprises an at least moderate fat content.

18. The method of claim 1, wherein the DTG or pharmaceutically acceptable salt thereof is within a pharmaceutical composition.

19. The method of claim 1, wherein the 3TC or pharmaceutically acceptable salt thereof is within a pharmaceutical composition.

20. The method of claim 13, wherein the DTG or pharmaceutically acceptable salt thereof and 3TC or pharmaceutically acceptable salt thereof are both within a 2-drug pharmaceutical composition.
